(19) United States
(12) Patent Application Publication
(10) Pub. No.: US 2026/0131037 A1
(43) Pub. Date: May 14, 2026

(54) MOBILE DECONTAMINATION SYSTEM

(71) Applicant: CLEAN WAKE LLC, Highland, UT (US)

(72) Inventor: Garrett Atwood, Highland, UT (US)

(21) Appl. No.: 19/390,047

(22) Filed: Nov. 14, 2025

Related U.S. Application Data
(60) Provisional application No. 63/720,410, filed on Nov. 14, 2024.

(51) Int. Cl. A61L 2/04; A61L 2/26; B08B 3/14; B08B 13/00; B60P 3/22; B62D 63/08; C02F 1/00; C02F 1/32

(52) U.S. Cl. CPC A61L 2/04; A61L 2/26; B08B 3/14; B08B 13/00; B60P 3/2205; B60P 3/2295; C02F 1/001; C02F 1/325; A61L 2202/122; A61L 2202/16; A61L 2202/17; B08B 2203/007; B62D 63/08; C02F 2201/008; C02F 2303/04

(57) ABSTRACT

Mobile decontamination systems are provided. In some cases, the systems include one or more vehicles, which have one or more decks (e.g., trailer-type vehicles). In some cases, one or more decontamination tanks is coupled to the deck. In some implementations, the decontamination tank is fluidically coupled to one or more decontamination components, such as heaters, sanitizers, filters, or other decontamination components. Additional implementations are described.

### CROSS-REFERENCE TO RELATED APPLICATION

This application claims priority to U.S. Provisional Patent Appl. No. 63/720,410 (Attorney Docket No. 31977.7), entitled “MOBILE DECONTAMINATION SYSTEM”, and which was filed on Nov. 14, 2024; the entire disclosure of which is incorporated herein.

### FIELD

The present disclosure relates to cleaning and decontamination, and more particularly to mobile systems with portable decontamination capabilities (e.g., for decontaminating field equipment, work sites, on-site systems, tools, vehicles, boats, trailers, helibuckets, or other systems and items that could benefit from a portable and robust decontamination system).

### BACKGROUND

Many types of equipment, work sites, vehicles, trailers, tools, and other locations or objects naturally become dirtied or contaminated through use (or non-use) over time. Contamination can include dust; dirt; particulate matter; microplastics; ash; bacteria, viruses, or other microbes; algae; spores; seeds; invasive plants; insect larvae or eggs (e.g., of invasive species); mollusks (e.g., quagga mussels), animals (e.g., barnacles, crabs, etc.); or any other contaminates that accumulate on or around an object or surface as a result of specific actions or due to the passage of time or as a result of exposure to such contaminants. Examples of equipment that can become contaminated include firefighting equipment (e.g., PPE, nozzles, hoses, valves, helibuckets, couplers, tools, water tanks, water scoopers, and other firefighting equipment); construction tools and implements; vehicles (automobiles, bicycles, watercraft, aircraft, spacecraft, trailers, or other vehicles) or vehicle parts; life preservers, machinery or machinery parts or equipment; electronic or electrical components; scientific equipment (e.g., instruments, tools); kitchen implements (e.g., silverware, dishes, and other kitchenware); buildings and building material; sports equipment; medical equipment; or any other type of tools, systems, or equipment that can become contaminated.

Currently, some types of equipment have limited methods of decontamination, often leading to use of contaminated equipment. This in turn can cause health problems, environmental damage, safety concerns, ecological harm, food chain issues, pollution, or other major issues. Alternatively, some types of equipment are decontaminated using unsafe or unsound methods, such that the decontamination process itself can cause any of the aforementioned issues or other problems. Moreover, some decontamination processes require specialized equipment that can be prohibitively expensive to acquire or operate. Additionally, many decontamination systems are large, bulky, unduly complex or expensive, or inconveniently located.

Thus, while some techniques currently exist that can be used to decontaminate equipment or work sites, challenges still exist, including those listed above. Accordingly, it would be an improvement in the art to augment or even replace current techniques with other techniques.

### DESCRIPTION OF THE FIGURES

The objects and features of the present disclosure will become more fully apparent from the following description and appended claims, taken in conjunction with the accompanying figures. Understanding that these figures depict only some embodiments of the disclosed systems and methods and are, therefore, not to be considered limiting in scope, the systems and methods will be described and explained with additional specificity and detail through the use of the accompanying figures in which:

FIG. 1 shows a side perspective view of a mobile decontamination system, in accordance with a representative embodiment of the disclosed systems and methods;

FIG. 2 shows a side perspective view of a first side of the mobile decontamination system, in accordance with a representative embodiment;

FIG. 3 shows a side perspective view of a second side of the mobile decontamination system of FIG. 2, in accordance with a representative embodiment;

FIG. 4 shows a plan view of the mobile decontamination system, in accordance with a representative embodiment;

FIG. 5 shows a side elevation view of the mobile decontamination system, in accordance with a representative embodiment;

FIG. 6 shows a back elevation view of the mobile decontamination system, in accordance with a representative embodiment;

FIGS. 7A-7C show basket components for use with the mobile decontamination system, in accordance with some representative embodiments;

FIG. 8 shows a cutaway view of a sanitizer for use with the mobile decontamination system, in accordance with a representative embodiment;

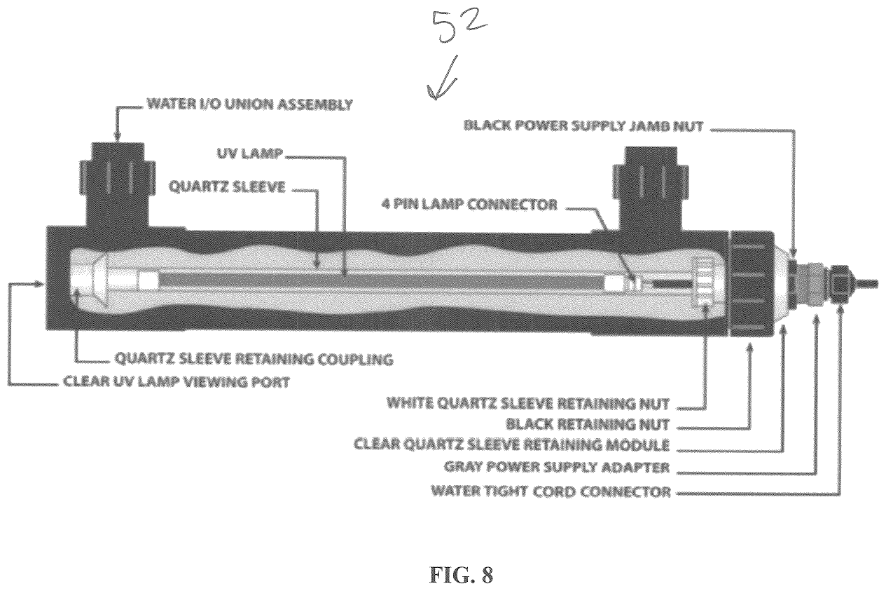

FIG. 9 shows a side perspective view of the mobile decontamination system with a container removed to reveal additional components, along with a detail of such components;

FIG. 10A shows a schematic view of the mobile decontamination system, in accordance with a representative embodiment;

FIG. 10B shows a perspective view of the mobile decontamination system, in accordance with a representative embodiment;

FIG. 11 shows a perspective view of a decontamination tank, in accordance with a representative embodiment; and

FIG. 12 shows a perspective view of a backflow valve testing system, in accordance with a representative embodiment.

### DETAILED DESCRIPTION

A description of embodiments will now be given with reference to the figures. It is expected that the present systems and methods may take many other forms and shapes. Hence the following disclosure is intended to be illustrative and not limiting, and the scope of the disclosure should be determined by reference to the appended claims.

The described mobile decontamination system 20 can include any suitable component necessary or desirable for specific or general decontamination tasks, such as cleaning, scrubbing, sanitizing, sterilizing, heating, spraying, soaking, purifying, purging, scouring, scrubbing, washing, disinfecting, deodorizing, or otherwise decontaminating equipment, surfaces, systems, objects, or areas of any kind. In some cases, the system optionally includes one or more vehicles 22.

Where the system 20 includes one or more vehicles 22, the vehicle can include any suitable vehicle or vehicular component allowing for transportation of the system. For example, some embodiments of the vehicle include one or more automobiles (e.g., trucks, vans, buses, pieces of heavy equipment, tractors, trains, or any other type of automobiles that are capable of functioning as the vehicle), motorcycles, bicycles, watercraft, aircraft, wagons, scooters, or other vehicles, but in some cases the vehicle includes one or more trailers configured to be pulled by another vehicle.

Some embodiments of the vehicle 22 include one or more decks 24. The deck can include any component configured to support one or more decontamination systems, auxiliary systems, cleaning components, storage components, or other objects or portions of the mobile decontamination system 20. In some cases, the deck includes one or more walking spaces, sitting spaces, observation decks, or other spaces for users. In some cases, the deck includes one or more mounts or other features that are designed to accommodate any of the components discussed herein.

While the vehicle 22 or the deck 24 thereof can be any suitable size, some embodiments of the vehicle (or the deck) have a width that generally conforms to traffic specifications, thereby allowing the vehicle to function as a road vehicle. In some cases, the width is between 2 feet and 20 feet (or any subrange thereof, such as between 5 feet and 15 feet, between 6 feet and 9 feet, or any other suitable subrange). In some cases, the vehicle (or the deck) has a length of between 5 feet and 55 feet (or any subrange thereof, such as between 10 feet and 30 feet, 15 feet and 25 feet, 18 feet and 22 feet, or any other suitable subrange).

The deck 24 can be formed of any suitable material, such as wood, metal, glass, plastic, carbon fiber, polymer material, or any other material suitable for use in connection with vehicles. According to some embodiments, the deck includes one or more additional safety features. In some cases, the safety features include a diamond plating, expanded metal sheeting, an anti-slip coating, or any other suitable feature to reduce the danger of walking on a surface that could potentially get wet.

Although the vehicle 22 can be configured to be transported in any suitable manner (e.g., having one or more engines, motors, wheels, tracks, skids, skis, runners, propellers, caterpillar tread, or any other suitable transportation mechanisms), some embodiments of the vehicle include one or more axle assemblies 26 (thereby providing one or more wheels 28 for the vehicle, allowing the vehicle to be driven or to be pulled as a trailer). Although various embodiments can include different types of axle assemblies, some embodiments include single axel. Some embodiments, however, include a dual axel with a maximum load capacity (e.g., gross vehicle weight (GVW)) of up to 500,000 lbs. (or any subrange thereof or other suitable load capacity, such as up to 20,000 lbs., 14,000 lbs., 10,000 lbs., or another suitable load capacity based on the weight of the system 20 and its intended functions). Where the vehicle has one or more wheels, any suitable wheels can be used (e.g., any size or type), but in some cases the wheels include heavy duty tires, off-road tires, snow tires, or other specializations allowing the system to be transported to remote locations or through difficult terrain.

Some embodiments of the vehicle 22 include one or more hitch assemblies 30. The hitch assembly can include anything that allows for the vehicle to be pulled like a trailer, such as one or more hitches 32. Although the hitch assembly can include any type of hitch (e.g., 5^{th} wheel hitch, weight distribution hitch, front mount hitch, gooseneck hitch, bumper hitch, rear receiver hitch, pintle hitch, receiver hitch, ball mount, trailer ball, trailer coupler, tow bar, or any other suitable coupling system), some implementations include a ball hitch (by way of non-limiting illustration, some embodiments include a 2- 5/16″ ball hitch or any other suitably sized hitch). Some embodiments of the hitch assembly include one or more hookups (e.g., electrical, water, hydraulic, mechanical, or other hookups for coupling brakes, lights, pipes, decontamination systems, chains, cables, or other components to another vehicle).

Some embodiments of the vehicle 22 include one or more spare tires, which in some cases are included with the hitch assembly 30.

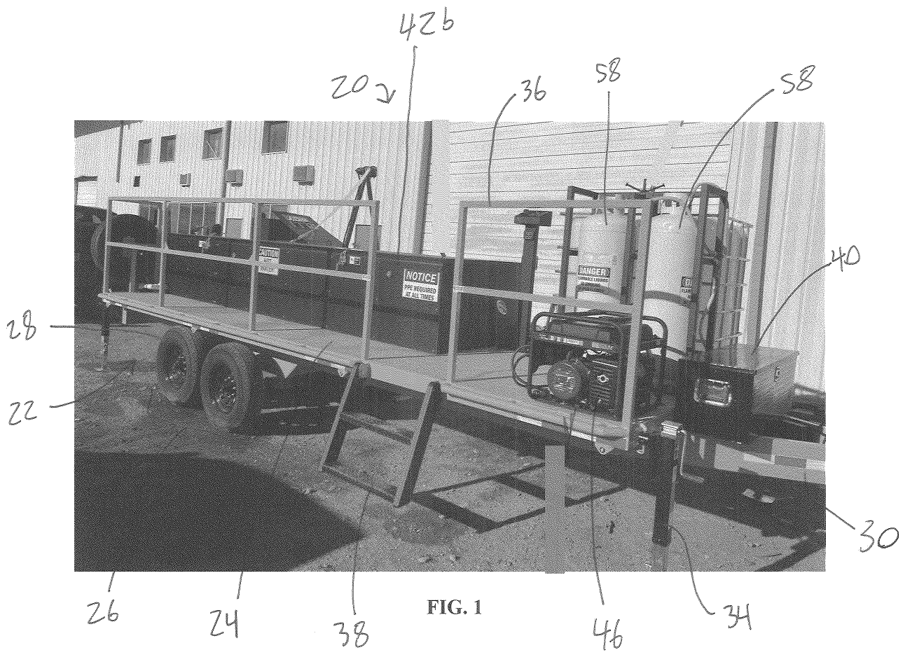

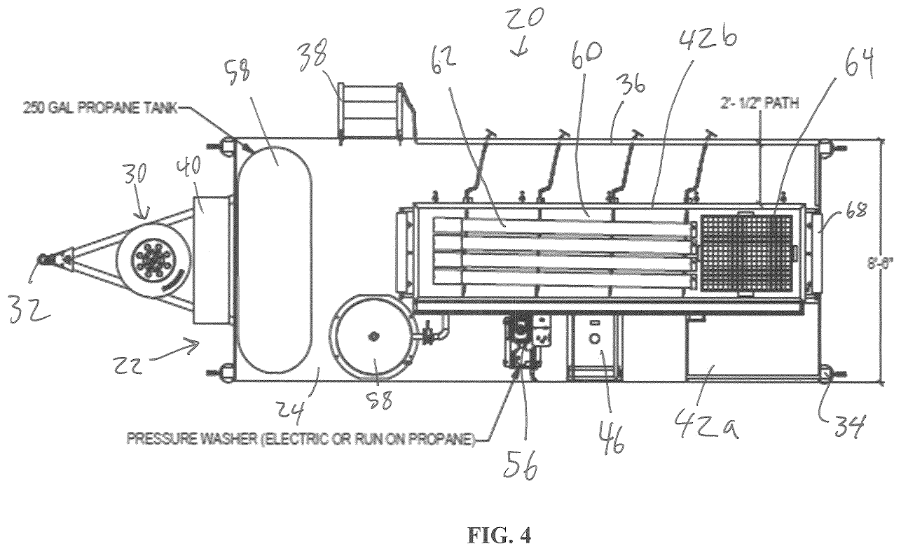

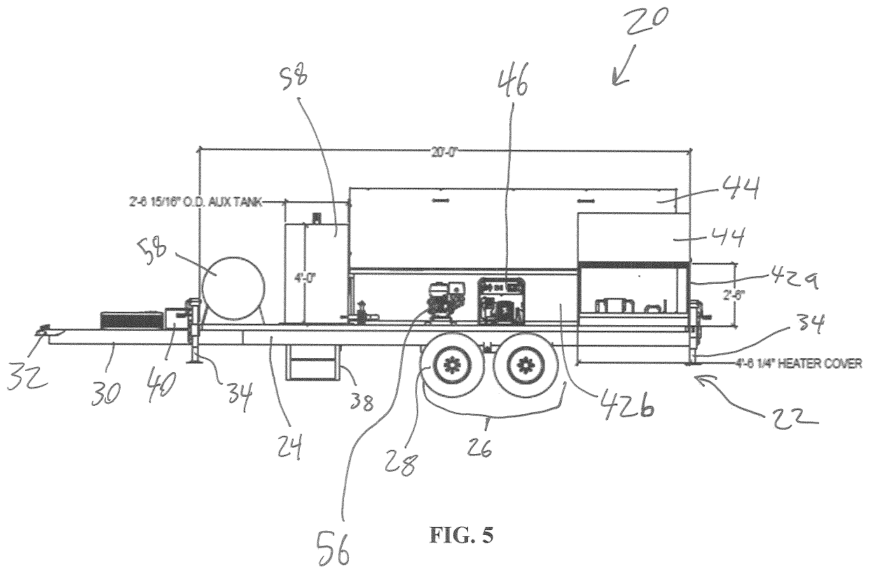

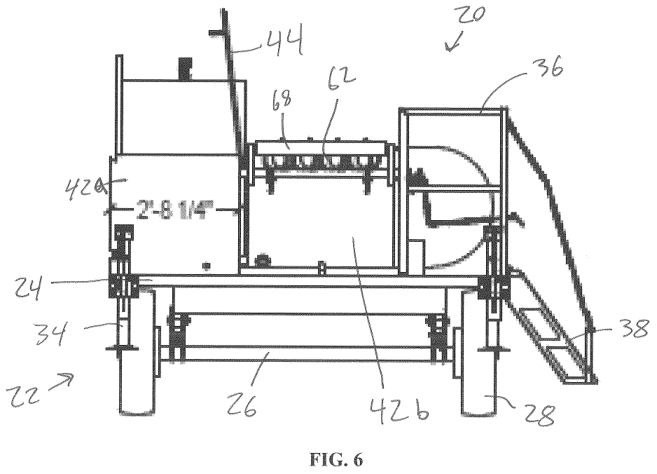

In some cases, the vehicle 22 includes one or more stabilizers 34. The stabilizer can include any component configured to stabilize, level, raise, or otherwise adjust the stability, angle, or height of the deck 24. For example, stabilizers can include one or more jacks (e.g., sidewind jacks, bottle jacks, scissor jacks, manual jacks, electric jacks, hydraulic jacks, or any other suitable jacks). By way of non-limiting illustrations, FIGS. 1-6 and 9 show examples of vehicles 22 with stabilizers 34 in the form of trailer jacks included at any suitable location with respect to (e.g., on the corners of) the deck 24, which jacks can be used to support and level the deck on the ground (therefore providing additional stability).

Some embodiments of the vehicle 22 optionally include one or more barriers 36 for safety or convenience. The barrier can include any wall, fence, railing, bars, poles, rails, frames, stanchions, railings, or any other suitable barrier (e.g., extending along all, part, or any portion or portions of a perimeter of the deck 24). In some cases, the barrier includes one or more access portions (e.g., doors, gate, window, breaks in the barrier, openings, or another access points). The barrier can have any suitable dimensions, such as a height of between 12 inches and 80 inches (or any subrange thereof, such as between 24 inches and 50 inches, 40 inches and 44 inches, or any other subrange). In some embodiments, an additional access feature 38 (e.g., a staircase, a step ladder, or another access feature) is included for easier access to the deck 24. By way of non-limiting illustration, FIG. 1 shows an access feature 38 in the form of a step or step ladder configured to fold up into a stowed position (where it functions as a portion of the barrier 36) or down into a deployed position (as shown in FIG. 1) such that it can be used to gain easier access to the deck 24.

Some embodiments include one or more utility boxes 40. The utility box can include any component capable of providing additional storage for tools, materials, cleaning supplies, maintenance supplies, or other components of any kind. The utility box can be formed of any suitable material (e.g., wood, metal, glass, plastic, carbon fiber, polymer material, or any other material), but in some cases it includes metal (e.g., aluminum). The utility box can also have any suitable dimensions. For example, any of the height, width, and length can be between 5″ and 150″ (or any suitable subrange thereof). By way of non-limiting illustration, the utility box may have a height of about 10″, a width of about 14″, and a length of about 50″ (in each case plus or minus 10%). In some embodiments, the utility box 40 is mounted on or adjacent to the axle assembly 26, but it may also be mounted (e.g., selectively, permanently, or semi-permanently coupled) on any other portion of the mobile decontamination system 20.

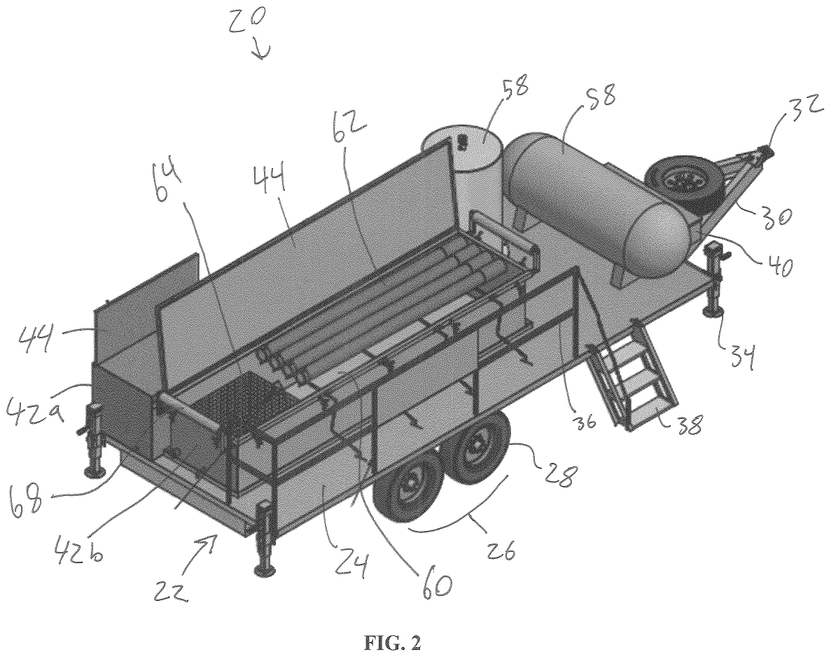

Some embodiments of the mobile decontamination system 20 include one or more functional components for decontamination. In some cases, some such components are mounted, mountable, or otherwise placed directly or indirectly on the vehicle 22 (e.g., by being bolted, welded, or otherwise selectively, permanently, or semi-permanently coupled to the deck 24 or another portion of the vehicle). In some cases, some such components are included within one or more containers 42 (which in turn are coupled to the deck, in some embodiments). In some cases, one or more components are both coupled directly to the deck and housed within a container (e.g., a bottomless container).

Where the mobile decontamination system 20 includes one or more containers 42, the containers can have any suitable configurations or dimensions that are useful for accommodating the corresponding components. For example, in some embodiments, the container is configured like a hollow box (which may have a rectangular prism shape, a cylindrical shape, a barrel shape, or any other shape). In some embodiments, the container includes one or more lids 44 that can be selectively opened to provide access to the contents of the container. The container can also be any suitable size. Indeed, in some cases, the container occupies between 5% and 90% of the area of the deck 24 (or any subrange thereof, such as between 5% and 20%, 15% and 50%, 20% and 40%, or any other subrange). In embodiments with multiple containers, the containers can also have any size with respect to each other (e.g., a first container can have between 5% and 500% of the volume of a second container (or vice versa), or any subrange thereof, such as between 5% and 15%, 10% and 20%, 15% and 30%, or any other suitable subrange). Indeed, in some embodiments, multiple containers are included that are sized or configured to accommodate different components, as discussed in additional detail below. By way of non-limiting illustration, FIGS. 2-6 show mobile decontamination systems 20 having a first container 42a and a second container 42b, each having a general box configuration, and each being configured to house one or more additional components, with the second container 42b being substantially larger than the first container 42a.

According to some embodiments, the system 20 includes one or more power sources. The power source can include any suitable power source (e.g., one or more batteries, plugs configured to receive power via one or more electrical outlets, solar panels, generators, capacitors, or other power sources), but some embodiments of the power source include one or more generators 46. The generator can include any suitable type of generator (e.g., standby generators, portable generators, inverter generators, induction generators, gasoline generators, natural gas generators, diesel generators, hydrogen generators, multiple fuel generators, or any other type of generators), but some embodiments include one or more propane generators or other gas-powered generators. In some cases, the generator uses a fuel (e.g., propane, gasoline, natural gas, diesel, or any other suitable fuel) that is also used by one or more other components of the system (thereby improving versatility and removing the need for additional types of fuel). By way of non-limiting illustration, FIG. 1 shows a system 20 having a generator 46 in the form of a propane/gas or diesel generator (e.g., Westinghouse™, 4650 watt, 1 30-amp and 1-20 amp circuit or any other generator). Although some embodiments include a power source within a container 42, some embodiments of the power source are not housed within a container (e.g., as shown in FIG. 1).

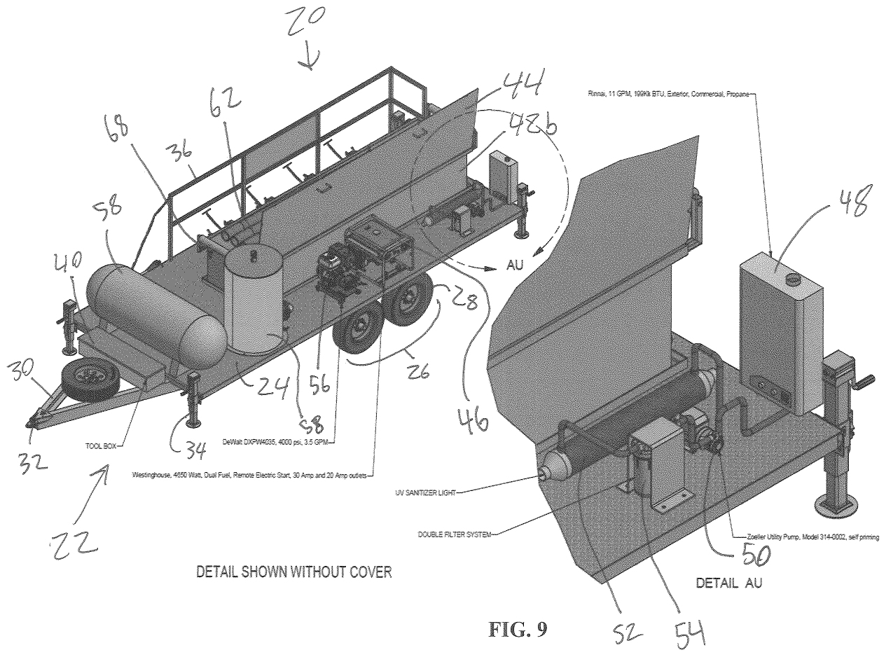

Some embodiments include one or more heaters 48. The heater can include any component capable of heating a liquid. For example, some embodiments include one or more tank heaters, tankless heaters, point-of-use heaters, solar heaters, hybrid heaters, boiler heaters, condensing heaters, heat pumps, induction heaters, coil heaters, microwave heaters, electric heaters, gasoline heaters, natural gas heaters, diesel heaters, electric heaters, or any other type of heater. By way of non-limiting illustration, FIG. 9 shows a system 20 having a heater 48 in the form of a tankless heater (e.g., Rinnai, 199K BTU, 11 GPM, commercial, exterior, propane or any other suitable type of heater). In some cases, the heater is powered by the power source (e.g., the generator 46), and in some cases the heater shares a common source of fuel with the power source (e.g., both the generator and the heater use propane (or another common fuel) to operate). In some cases, the heater is configured to use its own power source or fuel. In some embodiments, the heater is optionally housed within a container 42, such as a first container 42a.

Some embodiments include one or more pumps 50. Where the system 20 includes one or more pumps, the pumps can include any suitable type of pump useful for moving or circulating a fluid, such as one or more centrifugal pumps, positive displacement pumps, diaphragm pumps, peristaltic pumps, gear pumps, vane pumps, piston pumps, screw pumps, lobe pumps, axial flow pumps, jet pumps, submersible pumps, reciprocating pumps, rotary pumps, metering pumps, magnetic drive pumps, chemical pumps, air-operated pumps, multistage pumps, or any other suitable type of pump. By way of non-limiting illustration, FIG. 9 shows a system 20 having a pump 50 in the form self-priming utility pump (e.g., Armstrong, 182202-659, 115 VAC, 2.0 A, 32 GPM max, 230 Deg F. max). In some embodiments, the pump is optionally housed within a container 42, such as a first container 42a. In some embodiments, the pump is fluidically connected to the heater 48, and is configured to deliver fluid to or receive fluid from the heater (or both).

Some embodiments include one or more sanitizers 52. Where the system 20 includes one or more sanitizers, any components useful for sanitizing water (or any other suitable fluid) may be used, such as one or more chemical sanitizers, electrolysis sanitizers, mineral purifiers, filters, or other types of sanitizers. For example, some embodiments include one or more ultraviolet (UV) sanitizers configured to use UV light to sterilize water. By way of non-limiting illustration, FIG. 9 shows a system with a sanitizer 52 in the form of a UV sanitizing light. In some embodiments, the sanitizer is housed within a container 42, such as a first container 42a. In some cases, the sanitizer is fluidically connected to at least one of the pump 50 and the heater 48.

Furthermore, where the sanitizer 52 includes a UV sanitizer, the UV sanitizer can include any component for UV-powered sanitation. For example, some embodiments of the sanitizer 52 include one or more: valves or unions (e.g., water I/O union assemblies or other valve components); coupling components (e.g., retaining nuts, jamb nuts, retaining modules, adapters, connectors, or other coupling components); lamp components (e.g., UV lamps or bulbs, sleeves encompassing the lamps (such as a quartz sleeve), lamp connectors (e.g., a 4 pin lamp connector)); mixers; or other components for powering a UV sanitizer or otherwise allowing it to function as intended.

Some embodiments include one or more filters 54. Where the system 20 includes one or more filters, any components useful for filtering, cleaning, softening, or otherwise treating water may be used, such as one or more mechanical filters, carbon filters, particulate filters, reverse osmosis filters, water softeners, or other types of filtration or water treatment media configured to remove impurities, bacteria, sediment, minerals, seeds, eggs, spores, viruses, or any other undesirable matter from a liquid. By way of non-limiting illustration, FIG. 9 shows a system 20 with a filter 54 in the form of a double filtration system (e.g., a two-stage, 35 mesh and 100 mesh filtration system, or any other suitable filtration system). In some embodiments, the filter is housed within a container 42, such as a first container 42a. In some cases, the filter is fluidically connected to at least one of the sanitizer 52, the pump 50, and the heater 48.

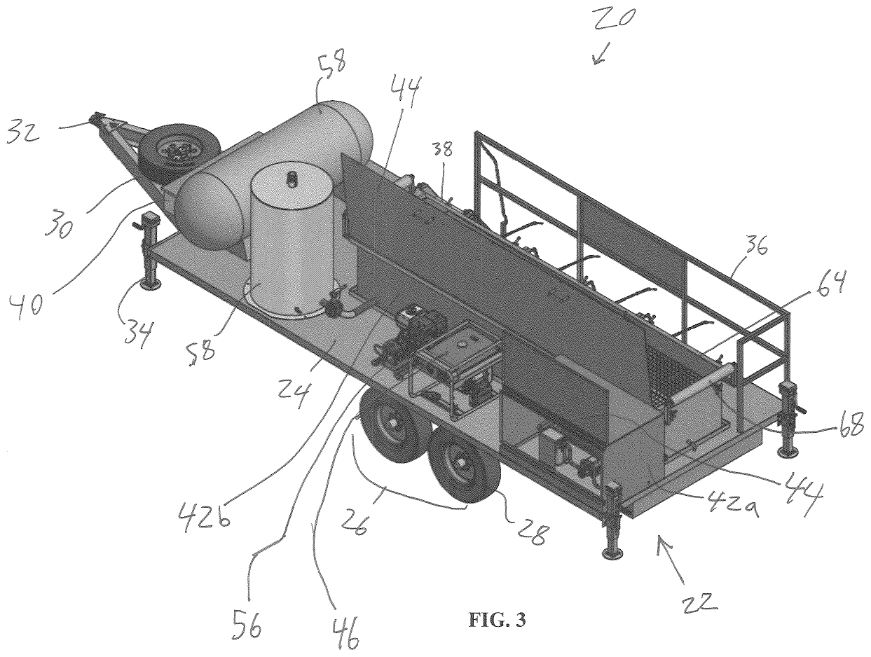

Some embodiments include one or more pressure washers 56. The pressure washer can include any component configured to deliver high-pressure water flow for cleaning, rinsing, scouring, spraying, or otherwise cleaning or decontaminating equipment. Although some embodiments of the pressure washer are at least partially housed within a container 42, some embodiments are not (e.g., they are open-access, allowing for relatively easy use and maintenance). Some embodiments of the pressure washer are configured to provide up to 10,000 pounds per square inch (psi) of pressure (or any subrange thereof, such as up to 5,000 psi, up to 4,000 psi, up to 3,000 psi, or up to any other suitable pressure). Some embodiments are configured to provide flow up to 10 gallons per minute (GPM), or any subrange thereof (e.g., up to 5 GPM, up to 3.5 GPM, or up to any other suitable flow rate. In some embodiments, the pressure washer is fluidically connected to at least one of the filter 54, the sanitizer 52, the pump 50, and the heater 48 (thereby having access to at least one of sanitized, filtered, and heated liquid). In some embodiments, the pressure washer is powered by the system's power source (such as the generator 46), utilizes its own fuel source, or utilizes a common fuel source (e.g., propane, diesel, gas, or another fuel) with the generator. By way of non-limiting illustration, FIGS. 3-5 and 9 show some embodiments of systems 20 having pressure washers 56 mounted to a deck 24 of a vehicle 22 for easy access (e.g., DeWalt™, 4000 psi, 3.5 GPM, gas powered pressure washer or any other suitable pressure washer).

According to some embodiments, the system 20 includes one or more storage tanks 58 (e.g., for holding a fluid, such as propane gas, natural gas, water, liquid gasoline, diesel, biofuel, cleaning solution, soap, detergent, or any other suitable fluid). For example, some embodiments of the system include a primary tank and one or more auxiliary tanks, while some embodiments of the system include multiple primary tanks configured to hold different fluids. The storage tanks can be any size suitable for holding the desired amounts of fluid. For example, in some embodiments one or more of the storage tanks has a capacity of between 2 gallons and 1,000 gallons (or any subrange thereof, such as between 100 gallons and 500 gallons, between 120 gallons and 250 gallons, or any other suitable capacity subrange). By way of non-limiting illustration, FIG. 2 shows a system 20 with two tanks 28 (e.g., a 250-gallon propane tank and a 150-gallon auxiliary fuel tank). The tanks can be formed of any suitable material (e.g., any plastic, metal, or other fluid-tight material), but in some cases they are formed of or comprise aluminum.

According to some embodiments, the system 20 includes one or more decontamination tanks 60. The decontamination tank can include any feature useful for decontaminating tools, materials, or other objects needing decontamination. For example, some embodiments of the decontamination tank include a large storage volume (e.g., between 2 gallons and 1000 gallons, or any subrange thereof, such as 500 gallons ±100 gallons, or any other suitable subrange). In some cases, the decontamination tank is configured to hold water or other decontamination liquid, which in some cases is heated (e.g., via the heater 48), sanitized (e.g., via the sanitizer 52), filtered (e.g., via the filter 54), or otherwise decontaminated or treated. Accordingly, in some embodiments, the decontamination tank is fluidically coupled to at least one other component (e.g., the heater, the pump 50, the filter, the sanitizer, the pressure washer 56, or another component). In some embodiments, water (or another liquid, such as a cleaning solution) is selectively circulated among the various components to provide for a constant cleaning process. As an example, the system 20 can prevent growth of algae in the decontamination tank via circulation through the sanitizer, or remove sediment via circulation through the filter. Similarly, the system can heat contents of the decontamination tank (in, before, or after the decontamination tank) to kill microbes or other organic matter in such contents.

Although the decontamination tank 60 can have any suitable feature, some embodiments of the decontamination tank are insulated to prevent heat loss. While the tank can be insulated in any suitable manner (e.g., via fiberglass insulation, foam board insulation, spray foam insulation, cellulose insulation, mineral wool insulation, reflective insulation, ceramic fiber, microporous (silica-based) insulation, firebrick, mica insulation, radiant barrier insulation, cotton (denim) insulation, polystyrene insulation, polyurethane insulation, rock wool insulation, aerogel insulation, loose-fill insulation, rigid foam insulation, structural insulated panels, or any other suitable type of insulation), some embodiments of the decontamination tank are double walled (e.g., with insulation disposed between the two walls) to prevent heat loss. Some embodiments of the decontamination tank are configured to maintain liquid at a high temperature (e.g., above 90, 125, 150, or 200 degrees Fahrenheit, or at any other suitable high temperature for decontamination (e.g., scalding or boiling temperature)). Indeed, in some embodiments, the system keeps a liquid (e.g., water) at a temperature between 90 and 210 degrees Fahrenheit, or at any subrange thereof. Indeed, in some embodiments, the system keeps a liquid at a temperature of between 95 degrees and 150 degrees Fahrenheit. In some embodiments, the decontamination tank is housed within a container 42, such as a second container 42b (which, in some cases, is fluidically coupled to a first container 42a or its contents). In some cases, the decontamination tank is integrally formed with the second container. In some cases, the entirety of the second container operates as the decontamination tank, whereas in some cases, the second container houses one or more additional components outside the decontamination tank.

According to some embodiments, the decontamination tank 60 includes one or more racks 62 configured to allow a user to more easily decontaminate equipment. The rack can include any component for facilitating the raising and lowering of equipment (including any suitable object) into the decontamination tank. For example, some embodiments of the rack include one or more surfaces (e.g., pipes, tubes, bars, sheets, baskets, racks, cables, chains, hooks, catches, or other surfaces) for holding equipment, and one or more movement mechanisms to allow the rack to descend into the liquid of the decontamination tank and ascend back out of it. In some cases, the movement mechanism includes one or more actuators configured to raise and lower the rack. In some cases, the actuator is powered by the generator 46. In some cases, the actuator is powered by another power source (e.g., propane, a solar cell, battery, combustible fuel, or another power source). The actuator can include any type of actuator (e.g., hydraulic actuators, pneumatic actuators, electromagnetic actuators, pneumatic linear actuators, electric actuators, mechanical actuators, manual actuators, diaphragm actuators, electromechanical actuators, thermal actuators, piezoelectric actuators, rotary actuators, linear actuators, solenoid actuators, voice coil actuators, shape memory alloy actuators, electromagnetic actuators, vacuum actuators, motors, winches, electronic winches, or any other type of actuator), but in some cases the actuator includes a hydraulic lift. By way of non-limiting illustration, FIG. 2 shows a decontamination tank 60 in a second container 42b having a rack 62 in the form of several tubular elements configured to collectively lower into the decontamination tank via one or more actuators (e.g., hydraulic lifts).

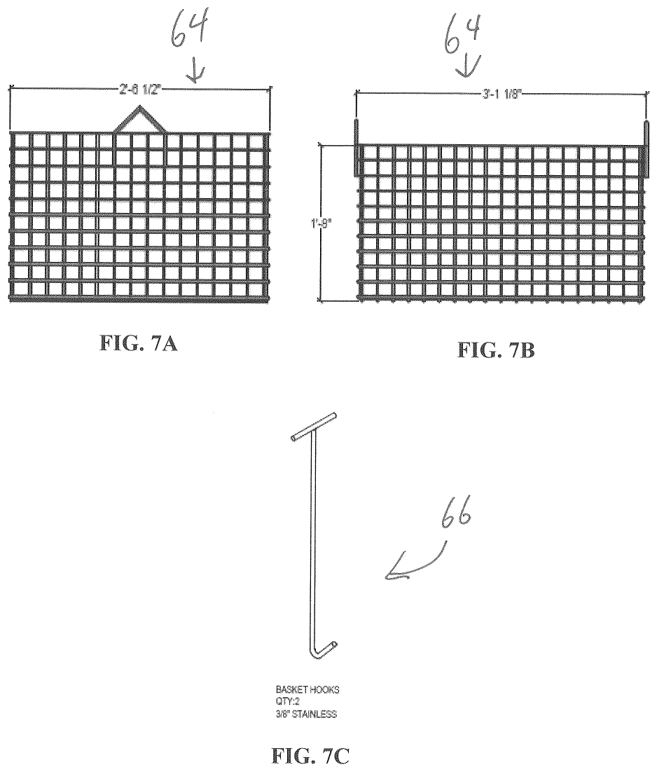

According to some embodiments, the decontamination tank 60 includes one or more baskets 64 configured to be lowered into the liquid of the decontamination tank to decontaminate the contents (for example, equipment that might otherwise float, or small equipment that could get lost if placed on the rack 62). The basket can include any suitable components or features that allow it to serve this purpose, but some embodiments include one or more wire meshes, perforated buckets, perforated containers, strainers, colanders, sieves, or any other suitable component that is cable of holding an object that is placed in the decontamination tank and that is capable of allow water (or any other suitable fluid) to leak from the component as it is removed from decontamination tank. The basket can be formed of any suitable material (e.g., any metal, polymer material, ceramic, natural material, synthetic material, or any other suitable material that is capable of withstanding the heat of the decontamination tank), but in some cases it includes stainless steel. It can also have any suitable dimensions, but in some cases it is configured to fit in a portion of the decontamination tank. For example, the basket can have a length of about 24″, a width of about 13″, and a depth of about 6″, in each case plus or minus 10%. Other dimensions allowing it to accomplish its intended function are also used in some embodiments. FIGS. 7A and 7B show illustrative embodiments of baskets 64 formed of or otherwise comprising wire meshes.

Some embodiments include one or more basket hooks 66 configured to facilitate removal of the baskets 66 from the decontamination tank 60. The basket hooks can include any component that aids an individual in removing the baskets from the decontamination tank. For example, FIG. 7C shows an example of a basket hook 66 having a J-shaped hook and a T-shaped handle.

Some embodiments of the decontamination tank 60 include one or more handles 68 for safety.

Some embodiments of the decontamination tank 60 include one or more lids 44 to serve as a safety protection, to increase pressure within the tank, to prevent or reduce evaporation from the tank, to keep fluid in the tank (e.g., to prevent water from splashing out when the system is moved), or to perform any other suitable function.

Some embodiments of the decontamination tank 60 include one or more drains. While the drains can be placed in any suitable locations, some embodiments of the drains are placed at low points in the decontamination tank to ensure that the tank will drain completely when the drains are opened. In some cases, the decontamination tank is configured to drain through the deck 24 (e.g., the drains are formed through the deck). In some cases, the drains are coupled to one or more drain hoses that route the draining water to a specific outlet, which can be formed in any portion of the vehicle 22 (e.g., near a rear of the vehicle).

Some embodiments of the decontamination tank 60 include one or more sensors. The sensors can be any suitable sensors, such as pH sensors, temperature sensors, fluid level sensors, ion sensors, turbidity sensors, conductivity sensors, residual chlorine sensors, dissolved oxygen sensors, chlorophyll sensors, green algae sensors, or other suitable types of sensors configured to help the system 20 maintain ideal or desired decontamination conditions. In some cases, the decontamination tank includes a first thermocouple and a second thermocouple disposed in different parts of the decontamination tank to ensure proper (e.g., sufficiently high, and even) heating throughout the tank. Similarly, some embodiments include one or more timers to help a user use the system for an adequate duration of decontamination.

Some embodiments of the decontamination tank 60 include one or more lid actuators configured to open and close the lid of the decontamination tank (e.g., the lid 44 of the second container 42b, or any other suitable lid). While such lid actuators can include any suitable actuators (e.g., any of the types of actuators discussed herein), some embodiments include one or more electronic winches configured to open and close the lid safely (thereby protecting users from the heat of the decontamination tank).

According to some embodiments, the system 20 includes one or more hoses or wires coupling various components together (e.g., hoses coupling one or more of the heater, the pump, the sanitizer, the filter, the pressure washer, the storage tank, the decontamination tank, and any other suitable components to each other; or wires coupling one or more of the generator, the heater, the pump, the sanitizer, the filter, the pressure washer, or any other suitable component to each other). In some embodiments, one or more of the hoses or wires are incorporated into the deck 24 (e.g., running beneath, within, around, or in any other suitable location with respect to an upper layer of the deck) such that one or more hoses or wires remain protected and out of the way. In some cases, a substantial portion of at least some of (or all of) the hoses and wiring is incorporated into the deck, such that less than 30%, less than 15%, less than 10%, or less than 5% of such hoses and wires are exposed.

Some embodiments of the system 20 are configured to accommodate firefighting equipment. For example, some embodiments of the decontamination tank 60 are sized and configured to accommodate draft hoses, footvalves, firebuckets, helmets, axes, gurneys, tools, and other components used in firefighting. Indeed, in some embodiments the decontamination tank is configured to receive and decontaminate objects that are placed in contact with water (e.g., hoses, pump parts, valves, couplings, etc.).

In some embodiments, the system 20, or any part thereof (e.g., the vehicle 22), is prepared according to particular quality specifications. In some cases, one or more surfaces is treated. For example, some embodiments involve sand blasting (e.g., SP6), powder deposition, or any other suitable treatment prior to, during, or after painting. Although any finish can be used, some embodiments utilize urethane paint. In some cases, paint is a particular color (e.g., forest service green) to correspond with a specific use (such as cleaning fire equipment after battling forest fires). In some cases, paint (or another coating or finish) is applied to a particular thickness (e.g., 4-6 mils, or another suitable subrange). Some embodiments include one or more safety notices (e.g., in some cases, a safety decal package is included).

The described systems and methods can be modified in any suitable manner. For instance, the system 20 can include one or more features for decontamination of specific tools, materials, or even vehicles. For example, some embodiments include one or more features for decontamination of watercraft (in some cases, adapted for other purposes, such as decontamination of other materials as discussed herein), such as any of the components discussed in U.S. Pat. No. 11,319,038, the contents of which are incorporated herein by reference in their entirety.

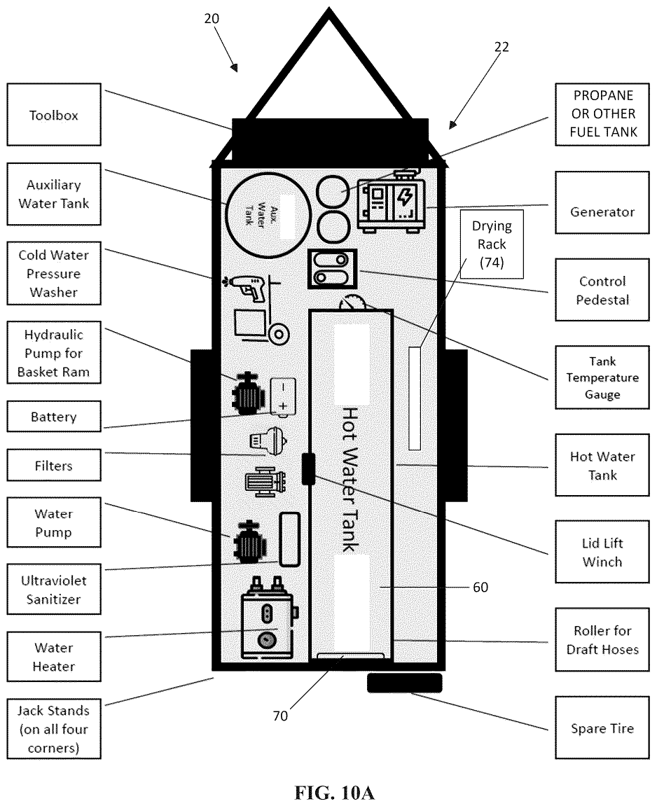

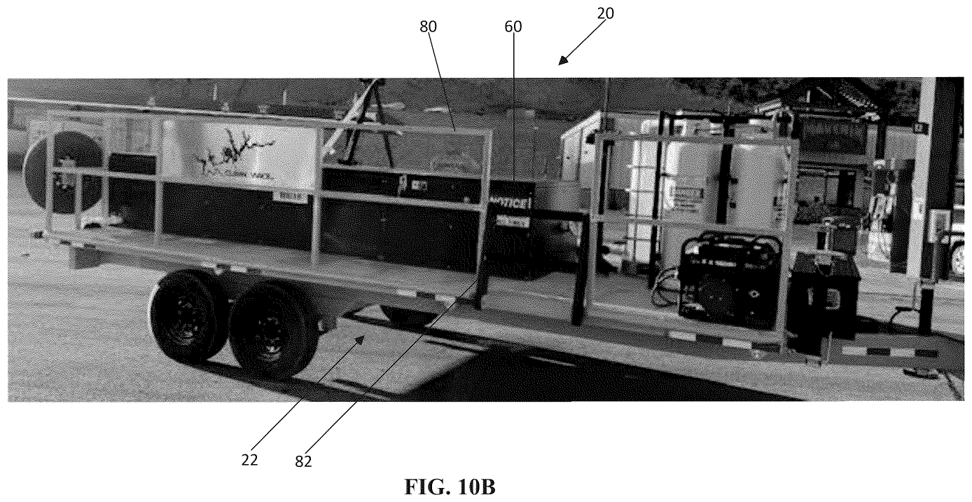

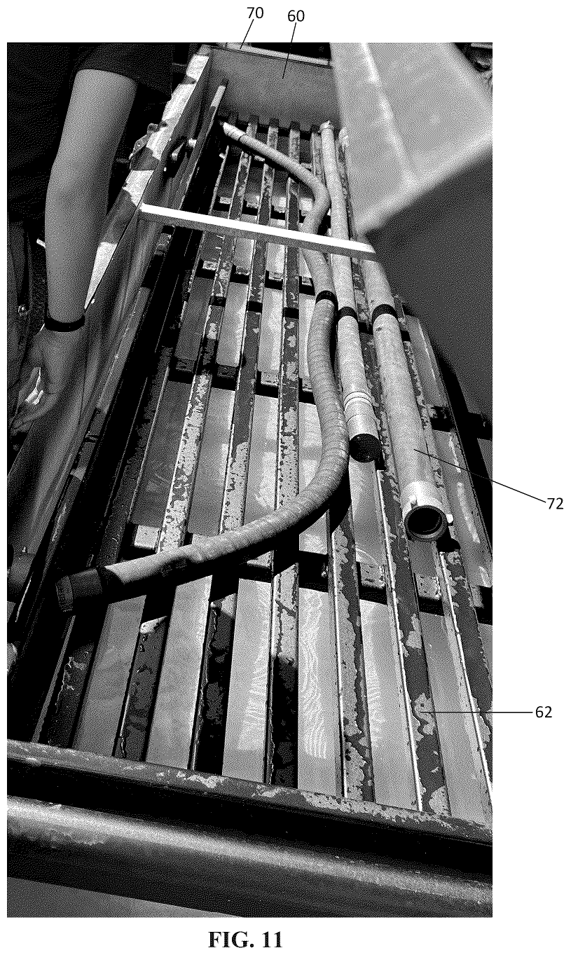

In accordance with some embodiments, the described system 20 comprises one or more rollers, roller systems, roller guides, hose roller guides, bearings, reels, guides, dispensers, slides, automatic retractors, hose carts, or any other suitable equipment that can facilitate hose movement or that protects one or more hoses from being scraped or otherwise damaged. By way of non-limiting illustration, FIGS. 10A and 11 show some embodiments in which the system 20 comprises one or more rollers 70 (e.g., cylindrical rollers) that allow one or more hoses or other objects to readily be fed into or connected to the decontamination tank 60 tank, to be placed on a rack 62, or to otherwise readily be moved with respect to a portion of the vehicle 22. Indeed, as shown in FIG. 11, some embodiments of the system 20 comprise one or more rollers 70 that allow hoses 72 that are on the ground (or in any other suitable location) to be moved across the roller 70 to easily be placed on the rack 62 such that the hoses 72 (or other objects) can easily be lowered into a hot water bath in the decontamination tank 60.

In accordance with some embodiments, the system 20 comprises one or more drying racks, slings, holders, hooks, catches, shelves, rails, frames, or other supports that are configured to hold one hoses 72, valves, or other objects as they dry or are otherwise stored. By way of non-limiting illustration, FIG. 10A shows some embodiments in which the system 20 comprises one or more drying racks 74.

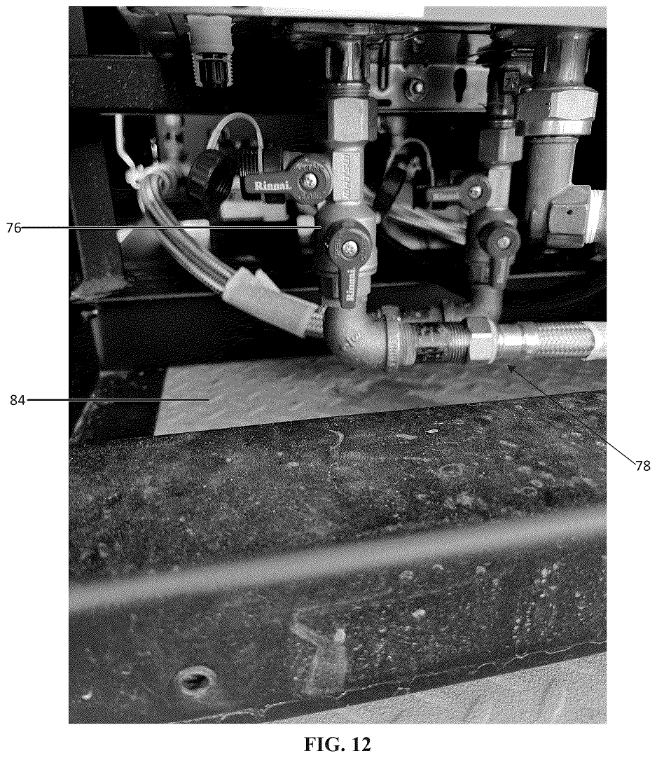

As another example of a suitable modification, in some embodiments, the system 20 comprises one or more backflow valve testing mechanisms that are configured to allow the use of pressurized water or another fluid to see if a valve is functioning properly. By way of non-limiting illustration, FIG. 12 shows some embodiments of such a backflow valve 76 and backflow valve testing system 78.

As still additional examples of suitable modifications, some embodiments of the described system 20 comprise one or more railings 80 (as shown in FIG. 10B), ladders 82 (as shown in FIG. 10B), antiskid surfaces 84 (as shown in FIG. 12), or any other safety component.

In addition to the aforementioned features, the described systems and methods can include any other suitable feature. For example, the decontamination system 20 of some embodiments is highly customizable. Accordingly, it can be configured specifically for use in connection with firefighting equipment, construction equipment, scientific equipment, or any other type of specialized equipment.

Thus, in some embodiments, some embodiments of the described system 20 can be taken to a point of use (e.g., a boat inspection site, a dock, a loading ramp, a decontamination site, etc.) where the system can be used to decontaminate objects (e.g., by placing such objects in hot water or any other suitable fluid in the decontamination tank 60, by spraying such objects with the pressure washer 56, or in any other suitable manner. Accordingly, some embodiments of the described system can readily and easily be used to decontaminate objects, even in locations that do not have municipal hook ups (e.g., for water, electricity, fuel, or gas).

Any and all of the components in the figures, embodiments, implementations, instances, cases, methods, applications, iterations, and other parts of this disclosure can be combined in any suitable manner. Additionally, any component can be removed, separated from other components, modified with or without modification of like components, or otherwise altered together or separately from anything else disclosed herein.

As used herein, the singular forms “a”, “an”, “the” and other singular references include plural referents, and plural references include the singular, unless the context clearly dictates otherwise. For example, reference to a fastener includes reference to one or more fasteners, and reference to actuators includes reference to one or more actuators. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object—regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

What is claimed is:

1. A mobile decontamination system comprising:
a vehicle comprising a deck;
a decontamination tank coupled to the deck; and
a component fluidically coupled to the decontamination tank, the component comprising at least one of a heater, a sanitizer, and a filter.

2. The mobile decontamination system of claim 1, wherein the system is configured to heat water for the decontamination tank such that the water has a temperature between 100 degrees Fahrenheit and 180 degrees Fahrenheit.

3. The mobile decontamination system of claim 1, further comprising a rack that is disposed in the decontamination tank, wherein the rack is configured to be raised and lowered in the decontamination tank.

4. The mobile decontamination system of claim 3, further comprising an actuator that is configured to raise and lower the rack relative to the decontamination tank.

5. A method for using a mobile decontamination unit, the method comprising:
heating water in the mobile decontamination unit, wherein the mobile decontamination unit comprises:
a vehicle comprising a deck;
a decontamination tank coupled to the deck and that is configured to hold the water; and
a component fluidically coupled to the decontamination tank, the component comprising at least one of a heater, a sanitizer, and a filter; and
placing an object in the decontamination tank such that the object is exposed to the water when a temperature of the water is between 100 degrees and 200 degrees Fahrenheit.

6. The method of claim 5, wherein the decontamination tank comprises a rack that is coupled to an actuator, and wherein the method further comprises placing the object on the rack and operating the actuator such that the rack and the object are lowered into the water in the decontamination tank.
